(19) United States
(12) Patent Application Publication
(10) Pub. No.: US 2026/0144567 A1
(43) Pub. Date: May 28, 2026

(54) ATHERECTOMY GUIDEWIRE BIAS EQUALIZATION FOR DIAMETER AND ELASTIC MODULUS

(71) Applicant: Boston Scientific Scimed, Inc., Maple Grove, MN (US)

(72) Inventors: Daniel Frank Massimini, Brooklyn Park, MN (US); Brice Lee Shireman, Maple Grove, MN (US); Michael Kaland, White Bear Lake, MN (US)

(73) Assignee: Boston Scientific Scimed, Inc., Maple Grove, MN (US)

(21) Appl. No.: 19/396,883

(22) Filed: Nov. 21, 2025

Related U.S. Application Data
(60) Provisional application No. 63/723,912, filed on Nov. 22, 2024.

(51) Int. Cl. A61B 17/3207; A61B 17/00; A61B 17/32

(52) U.S. Cl. CPC A61B 17/320758; A61B 2017/00526; A61B 2017/00867; A61B 2017/320052; A61B 2017/320775

(57) ABSTRACT

An atherectomy system may include an atherectomy device and a guidewire that is adapted to be used in combination with the atherectomy device. The atherectomy device includes a driveshaft and an atherectomy burr that is secured to the driveshaft. The guidewire includes a distal region having a distal region diameter ranging from 0.01 inches (0.254 millimeters) to 0.013 inches (0.33 millimeters) and a bending stiffness that emulates that of stainless steel having a diameter of 0.0077 inches (0.178 millimeters). The guidewire includes a proximal region having a proximal region diameter that is greater than the distal region diameter. The guidewire includes an intervening taper region. In some cases, the proximal region of the guidewire may have a bending stiffness that emulates that of stainless steel having a diameter of 0.009 inches (0.229 millimeters).

### CROSS-REFERENCE TO RELATED APPLICATIONS

This application claims the benefit of priority under 35 U.S.C. § 119 of U.S. Provisional Application No. 63/723,912, filed Nov. 22, 2024, the entire disclosure of which is hereby incorporated by reference.

### TECHNICAL FIELD

The present disclosure pertains to medical devices, and methods for manufacturing and using medical devices. More particularly, the present disclosure pertains to rotational medical devices, methods, and systems.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an atherectomy assembly. The atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen that extends through the atherectomy burr and that has a guidewire lumen diameter. The guidewire includes a distal region having a distal region diameter ranging from 0.01 inches (0.254 millimeters) to 0.013 inches (0.33 millimeters) and a bending stiffness that approximates that of stainless steel having a diameter of 0.0077 inches (0.178 millimeters), a proximal region having a proximal region diameter that is greater than the distal region diameter, and within 0.002 inches (0.0508 millimeters) of the guidewire lumen diameter, and an intervening taper region.

Another example may be found in an atherectomy assembly. The atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen that extends through the atherectomy burr and that has a diameter of 0.0145 inches (0.368 millimeters). The guidewire includes a distal region having a distal region diameter ranging from 0.01 inches (0.254 millimeters) to 0.013 inches (0.33 millimeters) and a bending stiffness that emulates that of stainless steel having a diameter of 0.0077 inches (0.178 millimeters), a proximal region having a proximal region diameter that is greater than the distal region diameter, and an intervening taper region.

Alternatively or additionally, the proximal region of the guidewire may have a bending stiffness that emulates that of stainless steel having a diameter of 0.009 inches (0.229 millimeters).

Alternatively or additionally, the distal region of the guidewire may have a bending stiffness of at least 1.5×10⁻⁵ Pascals (Pa) per unit length.

Alternatively or additionally, the distal region of the guidewire may have a bending stiffness of 1.5×10⁻⁵ Pascals (Pa) per unit length.

Alternatively or additionally, the proximal region of the guidewire may have a bending stiffness that ranges from 2.03×10⁻⁵ Pa/unit length to 4.75×10⁻⁵ Pa/unit length.

Alternatively or additionally, the guidewire may include a material having a Young's Modulus ranging from 4.35×10⁶ pounds per square inch (psi) (30 giga Pascals (GPa)) to 1.015×10⁷ psi (70 GPa).

Alternatively or additionally, the guidewire may have an overall length of 129.9 inches (330 cm) to 131.9 inches (335 cm) and the distal region of the guidewire may have a length ranging from 1 inch (2.54 cm) to 12 inches 30.5 cm).

Alternatively or additionally, the guidewire may include martensite nitinol.

Alternatively or additionally, the guidewire may include austenite nitinol.

Alternatively or additionally, the distal region of the guidewire may include nitinol and the proximal region of the guidewire may include stainless steel.

Alternatively or additionally, the guidewire may be formed of nitinol and may include a stainless sleeve extending over the proximal region of the guidewire.

Alternatively or additionally, the guidewire may further include a radiopaque component that extends through at least part of the guidewire.

Alternatively or additionally, the guidewire may further include a radiopaque material that is dispersed within the guidewire.

Another example may be found in an atherectomy assembly. The atherectomy assembly includes an atherectomy device and a guidewire that is adapted to be used in combination with the atherectomy device. The atherectomy device includes a driveshaft and an atherectomy burr that is secured to the driveshaft. The guidewire includes a distal region having a distal region diameter ranging from 0.0101 inches (0.257 millimeters) to 0.0125 inches (0.318 millimeters) and a bending stiffness that is at least 1.51×10⁻⁵ Pa/unit length and a proximal region having a proximal region diameter of 0.0135 inches (0.343 millimeters) or less and a bending stiffness that is in a range of 2.03×10⁻⁵ Pa/unit length to 4.75×10⁻⁵ Pa/unit length.

Another example may be found in an atherectomy assembly. The atherectomy assembly includes an atherectomy device and a guidewire that is adapted to be used in combination with the atherectomy device. The atherectomy device includes a driveshaft and an atherectomy burr that is secured to the driveshaft. The guidewire includes a distal region having a distal region diameter ranging from 0.0101 inches (0.257 millimeters) to 0.0125 inches (0.318 millimeters) and a bending stiffness that is 1.51×10⁻⁵ Pa/unit length and a proximal region having a proximal region diameter of 0.0135 inches (0.343 millimeters) and a bending stiffness that is in a range of 2.03×10⁻⁵ Pa/unit length to 4.75×10⁻⁵ Pa/unit length.

Alternatively or additionally, the atherectomy burr may include a guidewire lumen that extends through the atherectomy burr and that has a diameter of 0.0145 inches (0.368 millimeters).

Alternatively or additionally, at least the distal region of the guidewire may include a material having a Young's Modulus ranging from 4.35×10⁶ psi (30 GPa) to 1.015×10⁷ psi (70 GPa).

Alternatively or additionally, the guidewire may include nitinol.

Another example may be found in an atherectomy assembly. The atherectomy assembly includes an atherectomy device and a guidewire that is adapted to be used in combination with the atherectomy device. The atherectomy device includes a driveshaft and an atherectomy burr that is secured to the driveshaft. The atherectomy burr includes a guidewire lumen extending through the atherectomy burr having a guidewire lumen diameter. The guidewire includes a distal region having a distal region diameter of 0.0110 inches (0.279 millimeters) and a bending stiffness that emulates that of stainless steel having a diameter of 0.0077 inches (0.178 millimeters), a proximal region having a proximal region diameter within 0.002 inches (0.0508 millimeters) and a bending stiffness that emulates that of stainless steel having a diameter of 0.009 inches (0.229 millimeters), and an intervening taper region.

Another example may be found in an atherectomy assembly. The atherectomy assembly includes an atherectomy device and a guidewire that is adapted to be used in combination with the atherectomy device. The atherectomy device includes a driveshaft and an atherectomy burr that is secured to the driveshaft. The atherectomy burr includes a guidewire lumen extending through the atherectomy burr having a diameter of 0.0145 inches (0.368 millimeters). The guidewire includes a distal region having a distal region diameter of 0.0110 inches (0.279 millimeters) and a bending stiffness that emulates that of stainless steel having a diameter of 0.0077 inches (0.178 millimeters), a proximal region having a proximal region diameter of 0.0135 inches (0.343 millimeters) and a bending stiffness that emulates that of stainless steel having a diameter of 0.009 inches (0.229 millimeters), and an intervening taper region.

Alternatively or additionally, the guidewire may include martensite nitinol having a Young's Modulus of 4.35×10⁶ psi (30 GPa).

Alternatively or additionally, the guidewire may include austenite nitinol having a Young's Modulus of 1.015×10⁷ psi (70 GPa).

Alternatively or additionally, the guidewire may include a blend of martensite nitinol and austenite nitinol, the blend having a Young's Modulus of 7.25×10⁶ psi (50 GPa).

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram showing an illustrative atherectomy system;

FIG. 2 is a schematic diagram showing features of the illustrative atherectomy system of FIG. 1;

FIG. 3 is a schematic cross-sectional view of an illustrative atherectomy assembly that is usable as part of the illustrative atherectomy system shown in FIGS. 1 and 2;

FIG. 4 is a side view of an illustrative guidewire that may form part of the illustrative atherectomy assembly shown in FIG. 3;

FIG. 5 is a side view of an illustrative guidewire that may form part of the illustrative atherectomy assembly shown in FIG. 3;

FIG. 6 is a side view of an illustrative guidewire that may form part of the illustrative atherectomy assembly shown in FIG. 3;

FIG. 7 is a side view of an illustrative guidewire that may form part of the illustrative atherectomy assembly shown in FIG. 3;

FIG. 8 is a side view of an illustrative guidewire that may form part of the illustrative atherectomy assembly shown in FIG. 3;

FIG. 9 is a side view of an illustrative guidewire that may form part of the illustrative atherectomy assembly shown in FIG. 3;

FIG. 10 is a side view of an illustrative guidewire that may form part of the illustrative atherectomy assembly shown in FIG. 3; and

FIG. 11 is a side view of an illustrative guidewire that may form part of the illustrative atherectomy assembly shown in FIG. 3.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. Atheromatous and other vascular deposits may restrict blood flow and can cause ischemia in a heart of a patient, vasculature of a patient's legs, a patient's carotid artery, etc. Such ischemia may lead to pain, swelling, wounds that will not heal, amputation, stroke, myocardial infarction, and/or other conditions.

Atheromatous deposits may have widely varying properties, with some deposits being relatively soft and others being fibrous and/or calcified. In the latter case, the deposits may be referred to as plaque. Atherosclerosis occurs naturally as a result of aging, but may also be aggravated by factors such as diet, hypertension, heredity, vascular injury, and the like. Atherosclerosis may be treated in a variety of ways, including drugs, bypass surgery, and/or a variety of catheter-based approaches that may rely on intravascular widening or removal of the atheromatous or other material occluding the blood vessel. Atherectomy is a catheter-based intervention that may be used to treat atherosclerosis.

Atherectomy is an interventional medical procedure performed to restore a flow of blood through a portion of a patient's vasculature that has been blocked by plaque or other material (e.g., blocked by an occlusion). In an atherectomy procedure, a device on an end of a driveshaft is used to engage and/or remove (e.g., abrade, grind, cut, shave, etc.) plaque or other material from a patient's vessel (e.g., artery or vein). In some cases, the device on an end of the driveshaft may be abrasive and/or may otherwise be configured to remove plaque from a vessel wall or other obstruction in a vessel when the device is rotating and engages the plaque or other obstruction. In some cases, atherectomy involves using an abrasive atherectomy burr that is rotated at high speeds exceeding 100,000 revolutions per minute (RPM) in order to abrade plaque and other hardened materials from within the patient's vessel. Atherectomy burrs may be rotated at speeds exceeding 140,000 RPM, at speeds exceeding 180,000 RPM and even at speeds as high as 220,000 RPM. Atherectomy may include orbital atherectomy in addition to rotational atherectomy.

In some instances, an atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen that extends through the atherectomy burr. The guidewire lumen has a guidewire lumen diameter. The guidewire includes a distal region having a distal region diameter ranging from 0.01 inches (0.254 millimeters) to 0.013 inches (0.33 millimeters) and a bending stiffness that emulates that of stainless steel having a diameter of 0.0077 inches (0.178 millimeters). The guidewire includes a proximal region having a proximal region diameter that is greater than the distal region diameter, and within 0.002 inches (0.0508 millimeters) of the guidewire lumen diameter. The guidewire includes an intervening taper region. In some cases, the proximal region of the guidewire may have a bending stiffness that approximates that of stainless steel having a diameter of 0.009 inches (0.229 millimeters).

In some instances, an atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen that extends through the atherectomy burr. The guidewire lumen has a diameter of 0.0145 inches (0.368 millimeters). The guidewire includes a distal region having a distal region diameter ranging from 0.01 inches (0.254 millimeters) to 0.013 inches (0.33 millimeters) and a bending stiffness that emulates that of stainless steel having a diameter of 0.0077 inches (0.178 millimeters). The guidewire includes a proximal region having a proximal region diameter that is greater than the distal region diameter. The guidewire includes an intervening taper region. In some cases, the proximal region of the guidewire may have a bending stiffness that emulates that of stainless steel having a diameter of 0.009 inches (0.229 millimeters).

In some cases, the distal region of the guidewire may have a bending stiffness of at least 1.5×10⁻⁵ Pascals (Pa)/unit length. In some cases, the distal region of the guidewire may have a bending stiffness of 1.5×10⁻⁵ Pascals (Pa)/unit length. In some cases, the proximal region of the guidewire may have a bending stiffness that ranges from 2.03×10⁻⁵ Pa/unit length to 4.75×10⁻⁵ Pa/unit length. In some cases, the proximal region of the guidewire may have a bending stiffness of 2.81×10⁻⁵ Pa/unit length. In some cases, the guidewire may include a material having a Young's Modulus ranging from 4.35×10⁶ pounds per square inch (psi) (30 giga Pascals (GPa)) to 1.015×10⁷ psi (70 GPa). In some cases, the guidewire may have an overall length of 129.9 inches (330 cm) to 131.9 inches (335 cm) and the distal region of the guidewire may have has a length ranging from 1 inch (2.54 cm) to 12 inches 30.5 cm). In some cases, the guidewire may include multiple tapers, for example.

In some cases, the guidewire may include martensite nitinol. In some cases, the guidewire may include austenite nitinol. In some cases, the Young's Modulus of nitinol varies depending on the relative amounts of martensite nitinol and austenite nitinol. In some cases, the distal region of the guidewire may include nitinol and the proximal region of the guidewire may include stainless steel. In some cases, the guidewire may be formed of nitinol and may include a stainless sleeve extending over the proximal region of the guidewire. In some cases, the guidewire may further include a radiopaque component that extends through at least part of the guidewire. The guidewire may further include a radiopaque material that is dispersed within the guidewire. In some cases, other metals that may be used in forming the guidewire may include CoNiCr, MP35N (alloy of cobalt, nickel, chromium and molybdenum), variants of Hastelloy (nickel-based metals that are alloyed with chromium and molybdenum, and variants of Inconel (which are alloys of nickel containing chromium and iron).

In some instances, an atherectomy assembly includes an atherectomy device and a guidewire that is adapted to be used with the atherectomy device. The atherectomy device includes a driveshaft and an atherectomy burr that is secured to the driveshaft. The guidewire includes a distal region having a distal region diameter that ranges from 0.0101 inches (0.257 millimeters) to 0.0125 inches (0.318 millimeters) and has a bending stiffness that is in a range of 0.642×10⁻⁵ Pa/unit length to 3.51×10⁻⁵ Pa/unit length. The guidewire includes a proximal region having a proximal region diameter of 0.0135 inches (0.343 millimeters) and has a bending stiffness that is in a range of 2.03×10⁻⁵ Pa/unit length to 14.3×10⁻⁵ Pa/unit length.

In some cases, the atherectomy burr may include a guidewire lumen that extends through the atherectomy burr and has a diameter of 0.0145 inches (0.368 millimeters). In some cases, at least the distal region of the guidewire may include a material having a Young's Modulus ranging from 4.35×10⁶ psi (30 GPa) to 1.015×10⁷ psi (70 GPa). In some cases, the guidewire may include nitinol.

In some instances, an atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen extending through the atherectomy burr, the guidewire lumen having a guidewire lumen diameter. The guidewire includes a distal region having a distal region diameter of 0.0110 inches (0.279 millimeters) and a bending stiffness that emulates that of stainless steel having a diameter of 0.0077 inches (0.178 millimeters). The guidewire includes a proximal region diameter within 0.002 inches (0.0508 millimeter) of the guidewire lumen diameter and a bending stiffness that emulates that of stainless steel having a diameter of 0.009 inches (0.229 millimeters). An intervening taper region extends between the distal region and the proximal region.

In some instances, an atherectomy assembly includes an atherectomy device and a guidewire that is adapted to be used with the atherectomy device. The atherectomy device includes a driveshaft and an atherectomy burr that is secured to the driveshaft. The atherectomy burr includes a guidewire lumen extending through the atherectomy burr, the guidewire lumen having a diameter of 0.0145 inches (0.368 millimeters). The guidewire includes a distal region having a distal region diameter of 0.0110 inches (0.279 millimeters) and a bending stiffness that emulates that of stainless steel having a diameter of 0.0077 inches (0.178 millimeters). The guidewire includes a proximal region diameter of 0.0135 inches (0.343 millimeters) and a bending stiffness that emulates that of stainless steel having a diameter of 0.009 inches (0.229 millimeters). An intervening taper region extends between the distal region and the proximal region.

In some cases, the guidewire may include martensite nitinol having a Young's Modulus of 4.35×10⁶ psi (30 GPa). In some cases, the guidewire may include austenite nitinol having a Young's Modulus of 1.015×10⁷ psi (70 GPa). In some cases, the guidewire may include a blend of martensite nitinol and austenite nitinol, and the blend may have a Young's Modulus of 7.25×10⁶ psi (50 GPa).

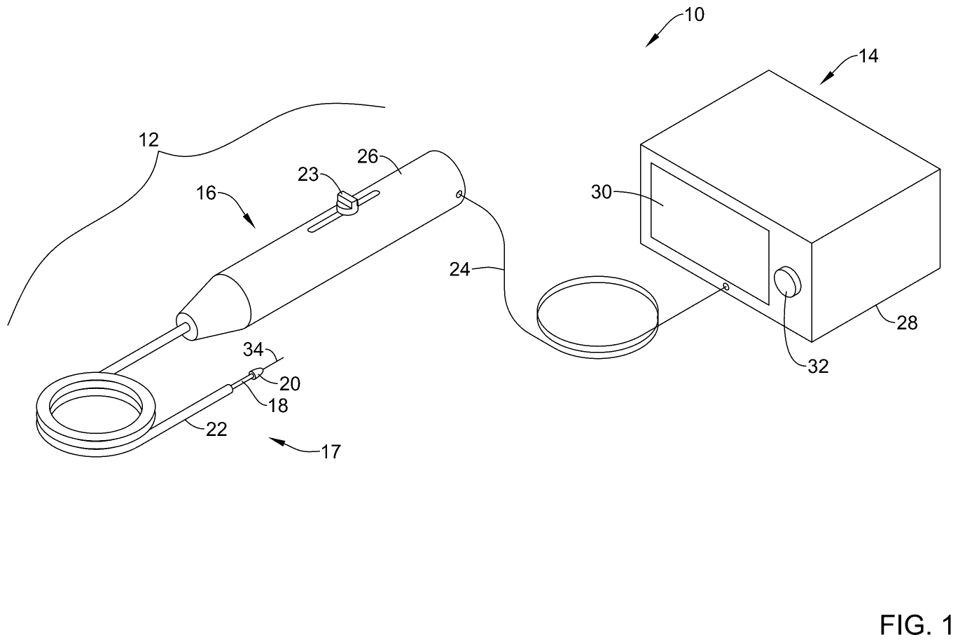

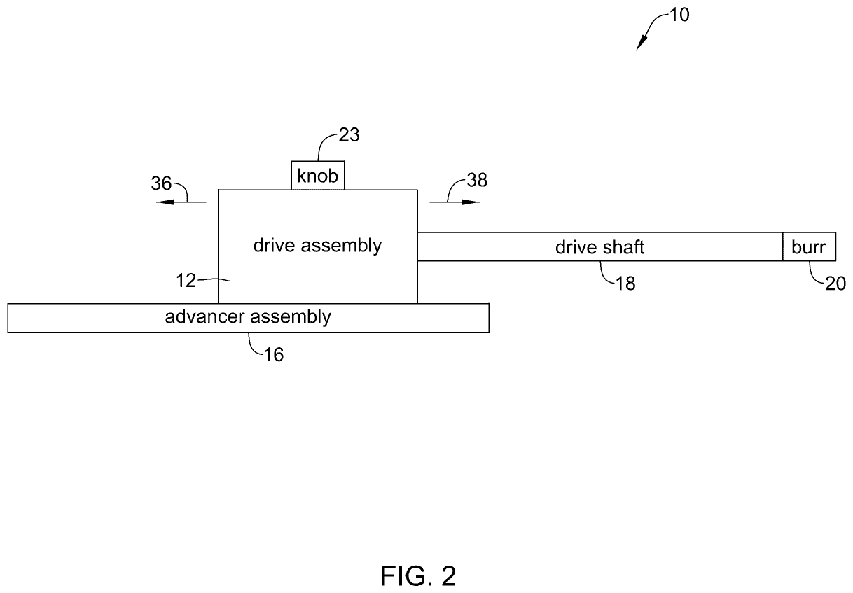

FIGS. 1 and 2 depict an atherectomy system 10. The atherectomy system 10 may be electrically driven, pneumatically driven and/or driven in one or more other suitable manners. Additional or alternative components to those illustrated and described herein may be utilized in the operation of the atherectomy system 10. The atherectomy system 10 may include a drive assembly 12 and a control unit 14 (e.g., a controller). The drive assembly 12 may include, among other elements, an advancer assembly 16 and a rotation assembly 17. Although the control unit 14 is depicted as being separate from the drive assembly 12 in FIG. 1, the functionality of the control unit 14 and the drive assembly 12 may be incorporated into a single component (e.g., in the advancer assembly 16 or other suitable single component).

The rotation assembly 17 may include a driveshaft 18 (e.g., an elongate member that may be or may include a flexible driveshaft or other suitable driveshaft), an atherectomy burr 20 and an elongate member 22 having a first end (e.g., a proximal end), a second end (e.g., a distal end), and a lumen extending from the first end to the second end for receiving the driveshaft 18. In some cases, the elongate member 22 may be an elongated tubular member. The driveshaft 18 may include a lumen extending therethrough (not shown) in order to accommodate a guidewire 34. In some cases, the guidewire 34 may be advanced through the vasculature to reach a desired treatment site, and then the driveshaft 18 (with the atherectomy burr 20 secured thereto) may be advanced over the guidewire 34. In some cases, the driveshaft 18 and the atherectomy burr 20 may be rotated over the guidewire 34 during use. The atherectomy burr 20 may have a rough or sharp surface, such that it is configured to grind, abrade, cut, shave, etc. plaque from a vessel wall or other obstruction in a vessel when it is rotated. In some cases, the atherectomy burr 20, the driveshaft 18 and the guidewire 34 may in combination be considered as being an atherectomy assembly that may be used in combination with the atherectomy system 10.

The advancer assembly 16 may include a knob 23, a housing 26, the drive assembly 12 and/or one or more other suitable components. In some instances, the drive assembly 12 may be or may include a motor (e.g., an electric motor, pneumatic motor, or other suitable motor) at least partially housed within the housing 26 and in communication with the knob 23, the driveshaft 18, and the control unit 14. In some cases, the motive force may not be disposed within the drive assembly 12, but may instead be remotely located, with a flexible drive cable extending from the motive force to the drive assembly 12. The knob 23 may be configured to advance along a longitudinal path to longitudinally advance the drive assembly 12 and the rotation assembly 17. The housing 26 may at least partially house the drive assembly 12 and the knob 23 may be at least partially accessible from an exterior of the housing 26.

In some instances, the drive assembly 12 is adapted to be translationally secured relative to an advancer assembly 16. In some instances, the advancer assembly 16 may be adapted to be fixed in space, such as being secured to a table, for example. In some instances, the advancer assembly 16 may be part of an advancer housing such as the housing 26. The drive assembly 12 may also be disposed within an advancer handle, for example, but is adapted to translate back and forth (left and right in the illustrated orientation) as indicated by arrows 36 and 38 in response to a user moving the knob 23 in the directions indicated by the arrows 36 and 38. In some instances, as the drive assembly 12 moves back and forth, the driveshaft 18 also moves correspondingly.

The drive assembly 12 may be coupled to the driveshaft 18 in a suitable manner including, but not limited to, a weld connection, a clamping connection, an adhesive connection, a threaded connection, and/or other suitable connection configured to withstand rotational speeds and forces. The driveshaft 18 may be formed from one or more of a variety of materials. For example, the driveshaft 18 may be formed from one or more of a variety of materials, including steel, stainless steel, other metal, polymer, and/or other suitable materials. The atherectomy burr 20 may have an outer perimeter which is equal to or larger than a distal diameter of the driveshaft 18 and/or the elongate member 22. Alternatively or in addition, the atherectomy burr 20 may have an outer perimeter which is smaller than a diameter of the driveshaft 18 and/or the elongate member 22. The atherectomy burr 20 may be coupled to the driveshaft 18. Where the driveshaft 18 has a first end portion (e.g., a proximal end portion) and a second end portion (e.g., a distal end portion), the atherectomy burr 20 may be coupled to the driveshaft 18 at or near the second end portion. In some cases, the atherectomy burr 20 may be located at or adjacent a terminal end of the second end portion of the driveshaft 18.

The drive assembly 12 and the control unit 14 may be in communication and may be located in or may have a same housing and/or located in or have separate housings (e.g., the advancer assembly housing 26 and a control unit housing 28 or other housings). Whether in the same housing or in separate housings, the drive assembly 12 and the control unit 14 may be in communication through a wired connection (e.g., via one or more wires in an electrical connector 24 or other suitable electrical connector) and/or a wireless connection. Wireless connections may be made via one or more communication protocols including, but not limited to, cellular communication, ZigBee, Bluetooth, Wi-Fi, Infrared Data Association (IrDA), dedicated short range communication (DSRC), EnOcean, and/or any other suitable common or proprietary wireless protocol, as desired.

Although not necessarily shown in FIG. 1, the drive assembly 12 may include and/or enclose one or more operational features. For example, among other features, the drive assembly 12 may include a motor (e.g., as discussed above and/or other suitable motor), rubber feet, control electronics, drive circuitry, etc.

The control unit 14, which may be separate from the drive assembly 12 (e.g., as shown in FIG. 1) or may be included in the drive assembly 12, may include several features. For example, as shown in FIG. 1, the control unit 14 may include a display 30 and a control knob 32 (e.g., a motor speed (e.g., RPM or other speed) adjustment knob or other control knob). Additionally or alternatively, the control unit 14 may include one or more other features for controlling the drive mechanism and/or other features of the drive assembly 12 (e.g., one or more drive mechanism states of the drive mechanism) including, but not limited to, a processor, memory, input/output devices, a speaker, volume control buttons, on/off power supply switch, motor activation switch, a timer, a clock, and/or one or more other features.

In some cases, the control unit 14 may include one or more drive load output control mechanisms for controlling an operation of the atherectomy system 10. In one example of a drive load output control mechanism that may be included in the control unit 14, the control unit 14 may include a mechanism configured to set and/or adjust an advancing load output (e.g., a rotational speed) and/or a retracting load output from the drive assembly 12. Additionally or alternatively, the control unit 14 may include other control and/or safety mechanism for controlling the operation of the atherectomy system 10 and mitigating risks to patients.

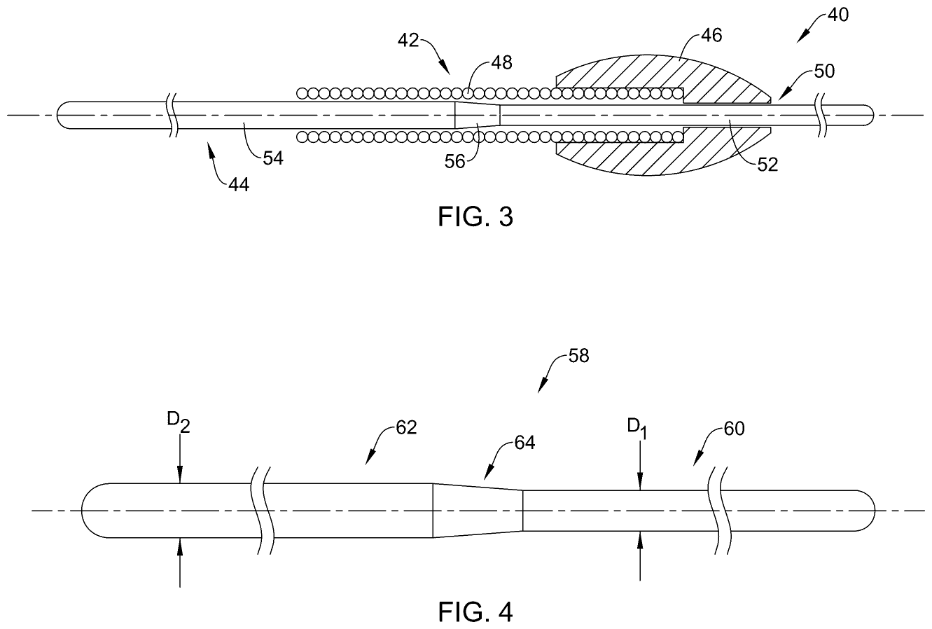

The atherectomy burr 20 may be coupled to the driveshaft 18 in any manner. For example, the atherectomy burr 20 may be coupled to the drive shaft 18 with an adhesive connection, a threaded connection, a weld connection, a clamping connection, and/or other suitable connection configured to withstand rotational speeds and forces. Similar to as discussed above with respect to the connection between the driveshaft 18 and the drive mechanism, as the driveshaft 18 and/or the atherectomy burr 20 may rotate at speeds between zero (0) RPM and 250,000 RPM or higher, the coupling between the driveshaft 18 and the atherectomy burr 20 may be configured to withstand such rotational speeds and associated forces. In some cases, the atherectomy burr 20 may be adhesively secured to the driveshaft 18. Because of the high speeds at which the driveshaft 18 and the atherectomy burr 20 are rotated, there is a desire for the atherectomy burr 20 to be accurately positioned relative to the driveshaft 18. As will be discussed with respect to FIGS. 3 through 11, the atherectomy burr 20 includes a cavity formed within the atherectomy burr 20 that accommodates a portion of the driveshaft 18 that extends into the cavity.

FIG. 3 is a schematic view of an illustrative atherectomy assembly 40 that includes an atherectomy device 42 and a guidewire 44 that is adapted to be used in combination with the atherectomy device 42. In some cases, the materials used in the construction of the guidewire 44, as well as the dimensions of the guidewire 44, may be selected in accordance with desired performance and the dimensions of the atherectomy device 42. The atherectomy device 42 includes an atherectomy burr 46 that may be considered as being an example of the atherectomy burr 20 and a driveshaft 48 that may be considered as being an example of the driveshaft 18 (see FIGS. 1 and 2 for the atherectomy burr 20 and the driveshaft 18). In some cases, the atherectomy burr 46 may have a guidewire lumen 50 that extends through the atherectomy burr 46. As an example, the guidewire lumen 50 may have a guidewire lumen diameter of 0.0145 inches (0.368 millimeters). The guidewire lumen diameter is appropriately dimensioned so that the atherectomy burr 46 may pass over the entirety of the guidewire 44.

In some cases, the guidewire 44 may include a distal region 52 having a distal region diameter that ranges from 0.0101 inches (0.257 millimeters) to 0.0125 inches (0.318 millimeters). The distal region 52 includes a bending stiffness that emulates that of stainless steel having a diameter of 0.0077 inches (0.178 millimeters). In some cases, the distal region 52 may have a bending stiffness that is 1.51×10⁻⁵ Pa/unit length or less. In some cases, the distal region 52 may have a bending stiffness that is 1.51×10⁻⁵ Pa/unit length. In some cases, the guidewire 44 includes a proximal region having a diameter that is within 0.002 inches (0.0508 millimeters) of the guidewire lumen diameter so that the atherectomy burr 46 is able to pass over the entirety of the guidewire 44. In some cases, the proximal region 54 has a proximal region diameter of 0.0135 inches (0.343 millimeters). In some cases, the proximal region 54 has a bending stiffness that emulates that of stainless steel having a diameter of 0.009 inches (0.229 millimeters). In some cases, the proximal region 54 may have a bending stiffness that is in a range of 2.03×10⁻⁵ Pa/unit length to 4.75×10⁻⁵ Pa/unit length. In some cases, the proximal region 54 may have a bending stiff that is 2.81×10⁻⁵ Pa/unit length. The guidewire 44 includes an intervening taper region 56 that is disposed between the distal region 52 and the proximal region 54, and has a diameter that tapers over a length between the distal region diameter of the distal region 52 and the proximal region diameter of the proximal region 54. As an example, the guidewire 44 may have an overall length ranging from 129.9 inches (330 cm) to 131.9 inches (335 cm). The distal region 52 may have a length ranging from 1 inch (2.54 cm) to 12 inches 30.5 cm).

In some cases, the guidewire 44 may have dimensions, including diameters, that are larger than those of the known guidewires that are commonly used in combination with atherectomy systems. Larger diameter guidewires can provide improvements in beneficial properties such as kink resistance, tactile feel and torque-ability but can negatively impact bending stiffness. In some cases, as will be discussed, particular combinations of guidewire dimension and material selection provides guidewires that are particularly well-suited for use as part of the atherectomy assembly 40. As an example, a particular stainless steel may have a Young's Modulus that is about 3.046×10⁷ psi (210 GPa). A martensite nitinol may have a Young's Modulus that is about 4.35×10⁶ psi (30 GPa). An austenite nitinol may have a Young's Modulus that is about 1.015×10⁷ psi (70 GPa). A blend of martensite nitinol and austenite nitinol may have a Young's Modulus that is about 7.252×10⁶ psi (50 GPa). In some cases, austenite nitinol has a body center cubic structure. In some cases, martensite nitinol has a monoclinic crystal structure. The particular crystalline structures can contribute to the shape memory properties of nitinol.

In some cases, bending stiffness may be considered as being proportional to the Young's Modulus (E), in accordance with the equation:

$$Bending Stiffness = \frac{E * I}{L} ,$$

where L is length and I is the moment of inertia, and is given (for a cylinder) by the equation:

$$I = \frac{π {D}^{4}}{64} ,$$

where D is diameter of the cylinder.

FIGS. 4 through 6 provide examples of illustrative guidewires that may be used as the guidewire 44 in providing improved atherectomy assemblies. FIG. 4 is a schematic view of an illustrative guidewire 58 that may be considered as being an example of the guidewire 44. The guidewire 58 includes a distal region 60 that has a diameter D₁, a proximal region 62 that has a diameter D₂, and an intervening tapered region 64 that has a diameter that tapers between the diameter D₁ and the diameter D₂. In some cases, the diameter D₁ may be in a range of 0.0101 inches (0.257 millimeters) to 0.0125 inches (0.318 millimeters). As an example, the diameter D₁ may be equal to 0.0110 inches (0.279 millimeters). In some cases, the diameter D₂ may be equal to 0.0135 inches (0.343 millimeters), which allows the atherectomy burr 46 (FIG. 3), which has a guidewire lumen 50 having a diameter of 0.0145 inches (0.368 millimeters), to be able to pass over the proximal region 62 of the guidewire 58.

In some cases, the distal region 60 may have a bending stiffness that is equivalent to the bending stiffness of a stainless steel wire having a diameter of 0.0077 inches (0.196 millimeters). In some cases, the distal region 60 may have a bending stiffness of 1.51×10⁻⁵ Pa/unit length. In some cases, the proximal region 62 may have a bending stiffness that is equivalent to the bending stiffness of a stainless steel wire having a diameter of 0.0094 inches (0.2286 millimeters). In some cases, the proximal region 62 may have a bending stiffness that is in a range of 2.81×10⁻⁵ Pa/unit length to 14.3×10⁻⁵ Pa/unit length. In some cases, the guidewire 58 may be formed of nitinol. As an example, the guidewire 58 may be formed of a blend of martensite nitinol and austenite nitinol having a Young's Modulus that is 7.252×10⁶ psi (50 GPa).

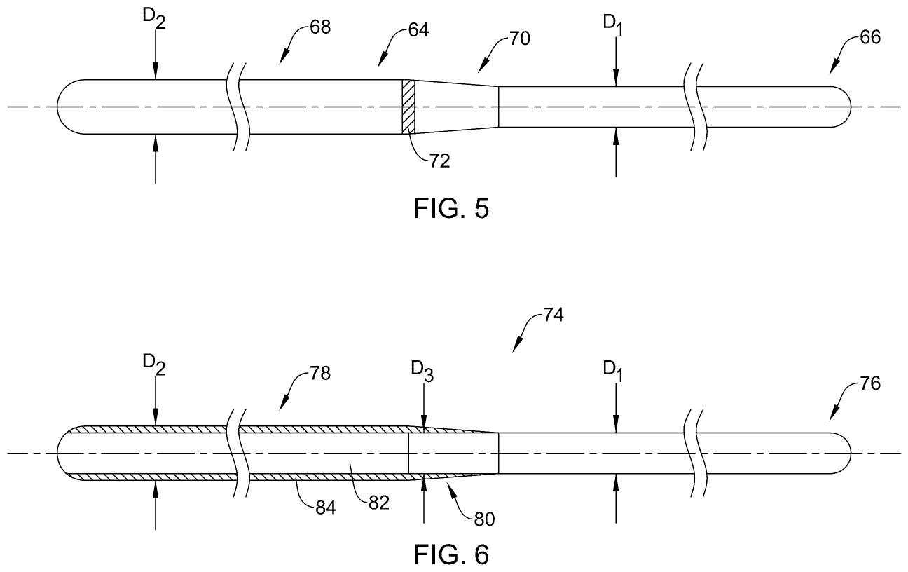

FIG. 5 is a schematic view of an illustrative guidewire 64 that may be considered as being an example of the guidewire 44. The guidewire 64 includes a distal region 66 that has a diameter D₁, a proximal region 68 that has a diameter D₂, and an intervening tapered region 70 that has a diameter that tapers between the diameter D₁ and the diameter D₂. In some cases, the diameter D₁ may be in a range of 0.0101 inches (0.257 millimeters) to 0.0125 inches (0.318 millimeters). As an example, the diameter D₁ may be equal to 0.0110 inches (0.279 millimeters). In some cases, the diameter D₂ may be equal to 0.0135 inches (0.343 millimeters), which allows the atherectomy burr 46 (FIG. 3), which has a guidewire lumen 50 having a diameter of 0.0145 inches (0.368 millimeters), to be able to pass over the proximal region 62 of the guidewire 64.

In some cases, the distal region 66 may have a bending stiffness that is equivalent to the bending stiffness of a stainless steel wire having a diameter of 0.0077 inches (0.196 millimeters). In some cases, the distal region 66 may have a bending stiffness of 1.51×10⁻⁵ Pa/unit length. In some cases, the proximal region 68 may have a bending stiffness that is equivalent to the bending stiffness of a stainless steel wire having a diameter of 0.0094 inches (0.239 millimeters). In some cases, the proximal region 68 may have a bending stiffness ranging from 2.03×10⁻⁵ Pa/unit length to 4.75×10⁻⁵ Pa/unit length. In some cases, the distal region 66 may be formed of nitinol while the proximal region 68 may be formed of stainless steel. As an example, the distal region 66 may be formed of a blend of martensite nitinol and austenite nitinol having a Young's Modulus that is 7.252×10⁶ psi (50 GPa) and the proximal region 68 may be formed of stainless steel having a Young's Modulus of 3.046×10⁷ psi (210 GPa).

In some cases, the distal region 66 and the intermediate region 70 may be secured to the proximal region 68 via a spin-welding process that results in a weld 72. As shown, the weld 72 is positioned between the intermediate region 70 and the proximal region 68. In some cases, the weld 72 may be positioned between the intermediate region 70 and the distal region 66, for example, depending on how much of the guidewire 64 is formed of nitinol and how much of the guidewire 64 is formed of stainless steel. Spin-welding is a process that allows dissimilar materials to be joined together.

FIG. 6 is a schematic view of an illustrative guidewire 74 that may be considered as being an example of the guidewire 44. The guidewire 74 includes a distal region 76 that has a diameter D₁, a proximal region 78 that has a diameter D₂, and an intervening tapered region 80 that has a diameter that tapers between the diameter D₁ and the diameter D₂. In some cases, the diameter D₁ may be in a range of 0.0101 inches (0.257 millimeters) to 0.0125 inches (0.318 millimeters). As an example, the diameter D₁ may be equal to 0.0110 inches (0.279 millimeters). In some cases, the diameter D₂ may be equal to 0.0135 inches (0.343 millimeters), which allows the atherectomy burr 46 (FIG. 3), which has a guidewire lumen 50 having a diameter of 0.0145 inches (0.368 millimeters), to be able to pass over the proximal region 78 of the guidewire 74.

In some cases, the guidewire 74 may have a core 82 that is formed of nitinol and a sleeve 84 that is formed of stainless steel and that extends over at least part of the core 82. The sleeve 84 may have an inner diameter that matches an outer diameter of the core 82. The core 82 may be considered as having a diameter D₃ that is equal to the diameter D₁. The sleeve 84 may have an outer diameter that provides the proximal region 78 with a diameter equal to D₂. The sleeve 84 may be secured to the core 82 in any suitable manner. In some cases, the distal region 76 may have a bending stiffness that is equivalent to the bending stiffness of a stainless steel wire having a diameter of 0.0077 inches (0.196 millimeters). In some cases, the distal region 76 may have a bending stiffness in a range of 2.03×10⁻⁵ Pa/unit length to 14.3×10⁻⁵ Pa/unit length, depending on the thickness of the sleeve 84 and the Young's Modulus of the core 82 and the sleeve 84. In some cases, the proximal region 78, as a result of including the sleeve 84, may have a bending stiffness that is equivalent to the bending stiffness of a stainless steel wire having a diameter of 0.0094 inches (0.239 millimeters).

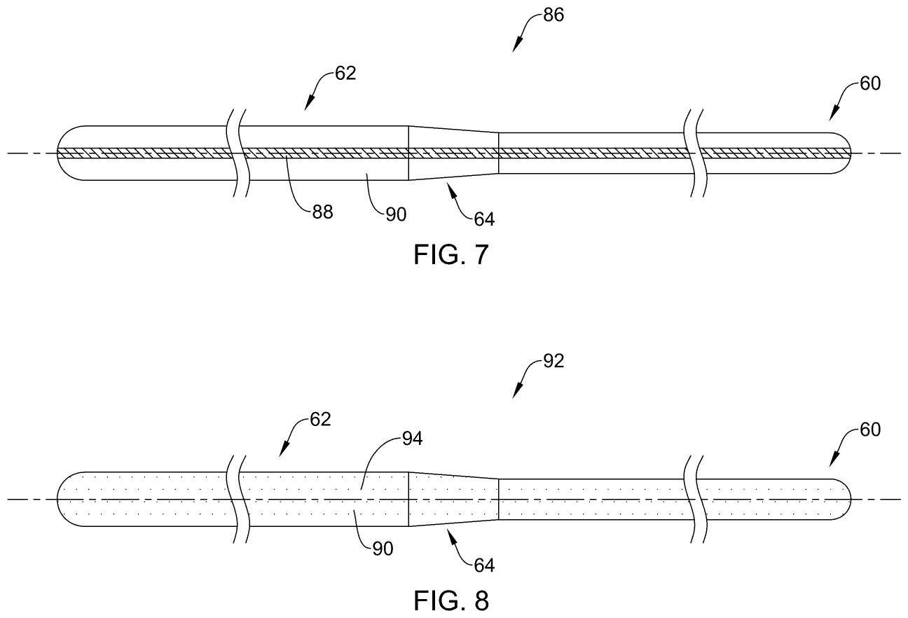

In some cases, the guidewires 44, 58, 64, and 74 described herein may be manufactured in a way that imparts at least some radiopacity to the guidewires 44, 58, 64, and 74 that allow improved visibility during fluoroscopic procedures. FIGS. 7 through 11 provide examples of providing radiopacity to the guidewire 44, 58, 64, and 74. FIG. 7 is a schematic view of an illustrative guidewire 86 that represents the guidewire 44 (FIG. 4) with the addition of a radiopaque core 88 that extends through a guidewire body 90. The radiopaque core 88 may be formed of any suitable radiopaque material such as tantalum, tungsten, gold or platinum. As an example, the radiopaque core 88 may be formed of tantalum. FIG. 8 is a schematic view of an illustrative guidewire 92 that represents the guidewire 44 (FIG. 4) with the addition of a radiopaque material 94 that is dispersed within the material forming the guidewire body 90. As an example, the guidewire body 90 may be formed of a mixture of nitinol and tantalum. In some cases, the guidewire body 90 may include 10 to 50 weight percent of the radiopaque material, with the balance being nitinol.

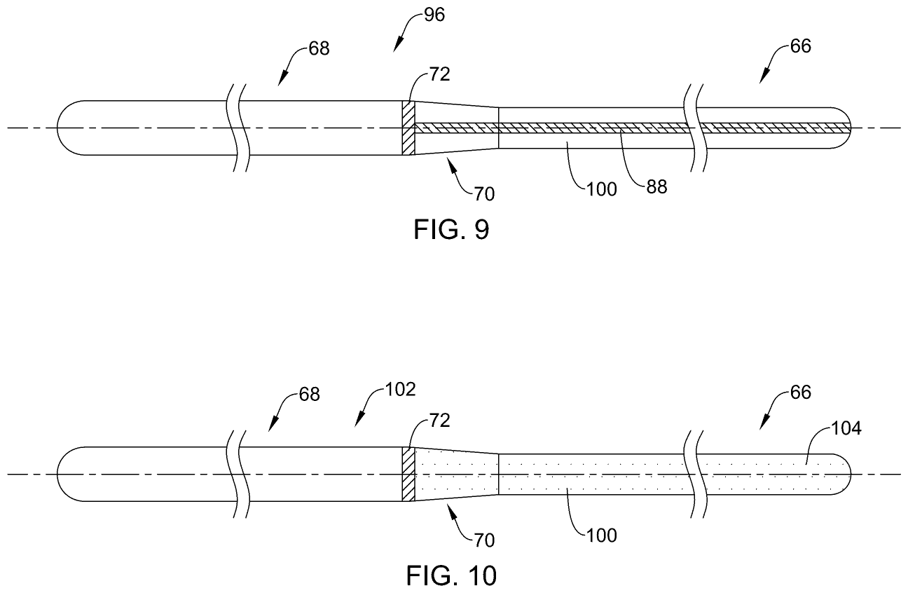

FIG. 9 is a schematic view of an illustrative guidewire 96 that represents the guidewire 64 (FIG. 5) with the addition of a radiopaque core 98 that extends through a guidewire body 100. In this case, the guidewire body 100 extends through the distal region 66 and the intervening tapered region 70 (both of which may be formed of nitinol) but does not extend into or through the proximal region 68 (which is formed of stainless steel). The radiopaque core 98 may be formed of any suitable radiopaque material such as tantalum, tungsten, gold or platinum. As an example, the radiopaque core 98 may be formed of tantalum. FIG. 10 is a schematic view of an illustrative guidewire 102 that represents the guidewire 64 (FIG. 5) with the addition of a radiopaque material 104 that is dispersed within the material forming the guidewire body 100. As an example, the guidewire body 100, which as noted extends through the intervening region 70 and the distal region 66 but not the proximal region 68, may be formed of a mixture of nitinol and tantalum.

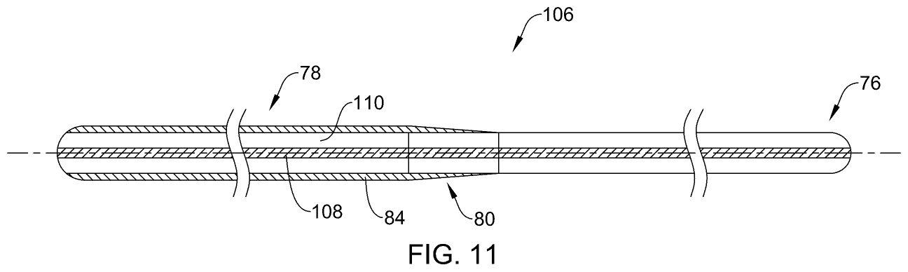

FIG. 11 is a schematic view of an illustrative guidewire 106 that represents the guidewire 86 (FIG. 7) with the addition of a radiopaque core 108 that extends through a guidewire body 110. The radiopaque core 108 may be formed of any suitable radiopaque material such as tantalum, tungsten, gold or platinum. As an example, the radiopaque core 108 may be formed of tantalum. In some cases, the radiopaque core 108 may extend through the distal region 76, the proximal region 78 and the intervening taper region 80. In some cases, the radiopaque core 108 may only extend through the distal region 76. In some cases, rather than a radiopaque core 108 extending through the guidewire body 110, the guidewire body 110 may instead include a radiopaque material that is dispersed throughout the guidewire body 110.

The materials that can be used for the various components of the devices and the various elements thereof disclosed herein may include those commonly associated with medical devices. In some instances, the devices described herein, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted into the devices and/or other elements disclosed herein. For example, the devices described herein, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An atherectomy assembly, comprising:
an atherectomy device including:
a driveshaft; and
an atherectomy burr secured to the driveshaft, the atherectomy burr including a guidewire lumen extending through the atherectomy burr, the guidewire lumen having a guidewire lumen diameter;
a guidewire adapted to be used in combination with the atherectomy device, the guidewire comprising:
a distal region having a distal region diameter ranging from 0.01 inches (0.254 millimeters) to 0.013 inches (0.33 millimeters), the distal region having a bending stiffness that approximates that of stainless steel having a diameter of 0.0077 inches (0.178 millimeters);
a proximal region having a proximal region diameter that is greater than the distal region diameter and within 0.002 inches (0.0508 millimeters) of the guidewire lumen diameter; and
an intervening taper region.

2. The atherectomy assembly of claim 1, wherein the proximal region of the guidewire has a bending stiffness that emulates that of stainless steel having a diameter of 0.009 inches (0.229 millimeters).

3. The atherectomy assembly of claim 1, wherein the distal region of the guidewire has a bending stiffness of at least 1.5×10⁻⁵ Pascals (Pa) per unit length.

4. The atherectomy assembly of claim 1, wherein the proximal region of the guidewire has a bending stiffness that ranges from 2.03×10⁻⁵ Pa/unit length to 4.75×10⁻⁵ Pa/unit length.

5. The atherectomy assembly of claim 1, wherein the guidewire comprises a material having a Young's Modulus ranging from 4.35×10⁶ pounds per square inch (psi) (30 giga Pascals (GPa)) to 1.015×10⁷ psi (70 GPa).

6. The atherectomy assembly of claim 1, wherein:
the guidewire has an overall length of 129.9 inches (330 cm) to 131.9 inches (335 cm); and
the distal region of the guidewire has a length ranging from 1 inch (2.54 cm) to 12 inches 30.5 cm).

7. The atherectomy assembly of claim 1, wherein the guidewire includes martensite nitinol.

8. The atherectomy assembly of claim 1, wherein the guidewire includes austenite nitinol.

9. The atherectomy assembly of claim 1, wherein:
the distal region of the guidewire comprises nitinol; and
the proximal region of the guidewire comprises stainless steel.

10. The atherectomy assembly of claim 1, wherein the guidewire is formed of nitinol and includes a stainless sleeve extending over the proximal region of the guidewire.

11. The atherectomy assembly of claim 1, wherein the guidewire further comprises a radiopaque component extending through at least part of the guidewire.

12. The atherectomy assembly of claim 1, wherein the guidewire further comprises a radiopaque material dispersed within the guidewire.

13. An atherectomy assembly, comprising:
an atherectomy device including:
a driveshaft; and
an atherectomy burr secured to the driveshaft;
a guidewire adapted to be used in combination with the atherectomy device, the guidewire comprising:
a distal region having a distal region diameter ranging from 0.0101 inches (0.257 millimeters) to 0.0125 inches (0.318 millimeters), the distal region having a bending stiffness that is at least 1.51×10⁻⁵ Pa/unit length; and
a proximal region having a proximal region diameter of 0.0135 inches (0.343 millimeters) or less, the proximal region of the guidewire has a bending stiffness that is in a range of 2.03×10⁻⁵ Pa/unit length to 4.75×10⁻⁵ Pa/unit length.

14. The atherectomy assembly of claim 13, wherein the atherectomy burr includes a guidewire lumen extending through the atherectomy burr, the guidewire lumen having a diameter of 0.0145 inches (0.368 millimeters).

15. The atherectomy assembly of claim 13, wherein at least the distal region of the guidewire comprises a material having a Young's Modulus ranging from 4.35×10⁶ psi (30 GPa) to 1.015×10⁷ psi (70 GPa).

16. The atherectomy assembly of claim 13, wherein the guidewire comprises nitinol.

17. An atherectomy assembly, comprising:
an atherectomy device including:
a driveshaft; and
an atherectomy burr secured to the driveshaft, the atherectomy burr including a guidewire lumen extending through the atherectomy burr, the guidewire lumen having a guidewire lumen diameter;
a guidewire adapted to be used in combination with the atherectomy device, the guidewire comprising:
a distal region having a distal region diameter of 0.0110 inches (0.279 millimeters) and a bending stiffness that emulates that of stainless steel having a diameter of 0.0077 inches (0.178 millimeters);
a proximal region having a proximal region diameter within 0.002 inches (0.0508 millimeters) of the guidewire lumen diameter and a bending stiffness that emulates that of stainless steel having a diameter of 0.009 inches (0.229 millimeters); and
an intervening taper region.

18. The atherectomy assembly of claim 17, wherein the guidewire comprises martensite nitinol having a Young's Modulus of 4.35×10⁶ psi (30 GPa).

19. The atherectomy assembly of claim 17, wherein the guidewire comprises austenite nitinol having a Young's Modulus of 1.015×10⁷ psi (70 GPa).

20. The atherectomy assembly of claim 17, wherein the guidewire comprises a blend of martensite nitinol and austenite nitinol, the blend having a Young's Modulus of 7.25×10⁶ psi (50 GPa).
